(19) United States
(12) Patent Application Publication
(10) Pub. No.: US 2026/0144814 A1
(43) Pub. Date: May 28, 2026

(54) METHOD FOR IMPROVING ATOPIC DERMATITIS BY USING A COMPOSITION COMPRISING LOW-MOLECULAR-WEIGHT FUCOIDAN

(71) Applicant: HI-Q MARINE BIOTECH INTERNATIONAL LTD., Taipei City (TW)

(72) Inventors: Hsien-Hung YANG, Taipei City (TW); Pin-Han Wang, Taipei City (TW); Pei-Yu Hsu, Taipei City (TW); I-Hsin Shih, Taipei City (TW); Huei-Ling Shih, Taipei City (TW); Chien Ting Hu, Taipei City (TW); Jr-Rung Lin, Taipei City (TW)

(21) Appl. No.: 19/007,823

(22) Filed: Jan. 2, 2025

(30) Foreign Application Priority Data
Nov. 22, 2024 (TW) 113145046

(51) Int. Cl. A61K 31/737; A61K 45/06; A61P 17/00

(52) U.S. Cl. CPC A61K 31/737; A61K 45/06; A61P 17/00

(57) ABSTRACT

The present invention is a method for improving atopic dermatitis, comprising administering to a subject in need thereof a composition comprising a low-molecular-weight fucoidan (LMF), wherein the weight-average molecular weight of the LMF is less than 3,000 Daltons. The composition of the present invention can effectively improve the area and severity of the lesion in patients with AD, as well as the itching symptoms (including insomnia symptoms caused by itching), and can significantly improve the SCORAD index of patients with AD. In addition, the composition of the present invention can reduce the patient's dependence on topical steroid ointments and/or oral antihistamines and reduce the AD-related immune parameters without increasing the biochemical indices of liver and kidney. Therefore, the composition of the present invention can be both effective and safe in the treatment of AD.

### CROSS-REFERENCE TO RELATED APPLICATION

Pursuant to 35 U.S.C. § 119 (a), this application claims the benefit of the priority to Taiwan Patent Application No. 113145046, filed on Nov. 22, 2024. The content of the prior application is incorporated herein by its entirety.

### BACKGROUND OF THE INVENTION

### 1. Field of the Invention

The present invention is a method for improving atopic dermatitis (AD), comprising administering a composition comprising low-molecular-weight fucoidan (LMF), or administering a medicine or food product prepared from the composition.

### 2. Description of the Prior Arts

AD is a chronic inflammatory skin disease associated with allergic constitution. Patients in their early stage will have erythema accompanied with leaked tissue fluid, blistering, and scabs, and patients in their late stage show scaling, cracking, and lichenification. Individuals at any ages are susceptible to AD, but this disease mainly occurs in children. Most people's conditions can be improved with age, but few people still suffer from continuing attacks or worsening even in their adulthoods. Patients with AD may develop allergies, such as having allergic rhinitis and asthma. Besides, AD can have crucial negative impact on work efficiency and daily activities.

The pathogenesis of AD is complex, mainly the epidermal barrier dysfunction and immune dysregulation resulting from the interaction between genetic factors and environmental factors. Patients with AD have to regularly use emollients, antihistamine, topical corticosteroids, topical calcineurin inhibitors, systemic immunosuppressants, phototherapies, and monoclonal antibody inhibitor. Among the options for the aforementioned medications, antihistamines primarily adopt the form of topical ointments as an antipruritic, suppressing the inflammation and itching of the affected areas, and patients with severe itching (such as itching causing insomnia symptoms) will also use oral antihistamines together. Currently, topical steroid drugs are the main current drugs of AD, but the long-term use will lead to skin thinning and discoloration. Due to the concerns about side effects, patients' adherence to corticosteroids and immunosuppressive drugs are poorer. Monoclonal antibody is a newly developed treatment, but it costs higher, so there are still restrictions on use. In addition, the medical costs of AD are huge, and the total cost burden in Taiwan is up to $3.61 billion New Taiwan Dollars in 2017 according to statistics.

Therefore, there is still an urgent need of an effective, safe, and low-cost treatment for AD to reduce the impact of AD on patients' health and life quality, and to reduce the financial burden on patients and the healthcare system.

### SUMMARY OF THE INVENTION

Given the shortcomings of the aforementioned prior art, one purpose of the present invention is providing a medicine for AD that simultaneously has effectiveness, safety, and low cost, comprising a composition of LMF (with a weight-average molecular weight of 3,000 Daltons or less).

The composition comprising LMF provided in the present invention can effectively improve the lesions, and area and severity thereof, and itching symptoms (including insomnia symptoms caused by itching) of patients with AD. In other words, it can significantly improve the SCORing atopic dermatitis (SCORAD) index of patients with AD. The composition of the present invention can reduce the frequency of patient application of commonly known topical steroid ointments and/or oral antihistamines, which means it can reduce the dependence on topical steroid ointments and/or oral antihistamines. In addition, the composition of the present invention can also reduce immune parameters related to AD, including immunoglobulin E (IgE), erythrocyte sedimentation rate (ESR), C-reactive protein (CRP), eosinophil percentage, CD8⁺ T cell percentage, and IFN-γ, and reduce the increase of IL-4. Therefore, the composition of the present invention is indeed effective when used in the treatment of AD. The composition of the present invention will not increase the biochemical indices of liver and kidney, and is safe when used in the treatment of AD.

Fucoidan is one of the key polysaccharides in brown algae, and there have been many studies on its therapeutic potential in recent years. The molecular weight of fucoidan ranges from 20,000 Daltons (Da) to 200,000 Da, or 400 Da to 5,000 Da, and the latter is called low-molecular-weight fucoidan (LMF). However, the clinical efficacy and safety of fucoidan are not fully understood at this stage.

To achieve the above purposes, the present invention provides a method for improving atopic dermatitis, comprising administering to a subject in need thereof a composition comprising LMF, wherein the weight-average molecular weight (Mw) of the LMF is 3,000 Da or less.

The present invention also provides a composition comprising LMF for use in preparing medicine for AD, wherein the weight-average molecular weight (Mw) of the LMF is 3,000 Da or less.

In some embodiments, the weight-average Mw of LMF is 160 Da to 3,000 Da, or 200 Da to 2,000 Da, or 500 Da to 1,000 Da, or 700 Da to 900 Da, or approximately 800 Da.

In some embodiments, the definition of AD includes: the patient's SCORAD index is equal to or larger than 16 (≥16). In some embodiments, the definition of AD further includes: itchy skin accompanied by 3 or more of the following situations: a) a history of itchiness in skin creases; b) a history of asthma and hay fever; c) a history of general dry skin in the past year; d) childhood onset at the age of two years or below; and e) visible flexural dermatitis. In some embodiments, the definition of AD further includes: the patient is aged between 4 and 60 years.

In some embodiments, an effective amount of LMF is as follows: for those weighing more than 40 kilograms (kg), the effective amount is 4.0 grams (g)/day; for those weighing 20 kg to 40 kg, the effective amount is 3.0 g/day; for those weighing 10 kg to less than 20 kg, the effective amount is 2.0 g/day; for those weighing less than 10 kg, the effective amount is 1.0 g/day. In some embodiments, the LMF comprises fucose monomers modified with sulfate groups; wherein, based on the total weight of the LMF as 1 g, the content of fucose is 200 micromoles (μmol) to 300 μmol, or 200 μmol to 250 μmol; and, based on the total weight of the LMF, the content of sulfate groups is 5% to 45% by weight, 5% to 40% by weight, 10% to 45% by weight, 10% to 40% by weight, 15% to 45% by weight, or 15% to 40% by weight.

In some embodiments, the medicine or food product comprises the composition and at least one pharmaceutically acceptable excipient. In some embodiments, the pharmaceutically acceptable excipient comprises a microcrystalline cellulose, water, or a combination thereof. In some embodiments, the microcrystalline cellulose comprises microcrystalline a-cellulose.

In some embodiments, the medicine is in an oral form. In some embodiments, the medicine is in a solid dosage form or a liquid dosage form. In some embodiments, the solid dosage form is powder, a granule, a tablet, or a capsule. In some embodiments, the liquid dosage form is a solution, a suspension, an elixir or a syrup.

In some embodiments, compared with pre-administration, the composition can reduce the objective indicators of SCORAD index after administration for a month. In some embodiments, compared with pre-administration, the composition can reduce the lesion area scores of SCORAD index by 25% or more, 30% or more, or 35% or more. In some embodiments, compared with pre-administration, the composition can reduce the lesion severity scores of SCORAD index by 15% or more, or 20% or more. In some embodiments, compared with pre-administration, the composition can reduce the subjective indicator scores of SCORAD index by 15% or more, or 20% or more. In some embodiments, compared with pre-administration, the composition can reduce the total scores of SCORAD index by 15% or more, or 20% or more.

In some embodiments, compared with pre-administration, the composition can reduce the subjective indicator of SCORAD index after used for a month. In some embodiments, compared with pre-administration, the composition can further reduce itching sensation or reduce insomnia symptoms caused by itching, thereby reducing the frequency of topical steroid ointment application by 40% or more, or 45% or more. In some embodiments, compared with pre-administration, the composition can further reduce itching sensation or reduce insomnia symptoms caused by itching, thereby reducing the frequency of oral antihistamine application by 25% or more, or 30% or more.

In some embodiments, compared with pre-administration, the composition can reduce the frequency of topical steroid ointment application and/or oral antihistamine application. In some embodiments, compared with pre-administration, the composition can reduce the frequency of topical steroid ointment application by 5% or more, or 7% or more. In some embodiments, compared with pre-administration, the composition can increase the number of people no longer using topical steroid ointments and/or oral antihistamines by 10% or more, 12% or more, or 15% or more.

In some embodiments, the composition can reduce the level of some AD-related immune parameters. In some embodiments, compared with pre-administration, the composition can reduce the IgE concentration in serum by 5% or more, or by 7% or more, or by 8% or more. In some embodiments, compared with pre-administration, the composition can reduce the ESR by 12% or more, or by 15% or more. In some embodiments, compared with pre-administration, the composition can reduce the CRP concentration in serum by 35% or more, or by 40% or more, or by 45% or more. In some embodiments, compared with pre-administration, the composition can reduce the eosinophil percentage by 15% or more, or by 20% or more. In some embodiments, compared with pre-administration, the composition can reduce the IFN-γ concentration in serum by 15% or more, or by 20% or more. In some embodiments, compared with pre-administration, when not administering this composition, the IL-4 concentration in serum of patients with AD will increase by 35% or more, or by 50% or more, or by 65% or more. However, the composition can reduce the increased concentration of IL-4 in serum to keep the concentration at the same level, or, to reduce the increased IL-4 concentration in serum by 15% or less.

In some embodiments, the composition further comprises a therapeutically effective amount of an oral antihistamine. In some embodiments, the oral antihistamine can be first-generation antihistamine or second-generation antihistamine. In some embodiments, the first-generation antihistamine may be chlorpheniramine or diphenhydramine. In some embodiments, the second-generation antihistamine may be cetirizine, loratadine, or fexofenadine. In some embodiments, the oral antihistamine is chlorpheniramine, with a therapeutically effective amount of 2 micrograms (mg) to 4 mg (single dose, suitable for children and adults, total daily dose not exceeding 12 mg to 24 mg). In some embodiments, the oral antihistamine is diphenhydramine, with a therapeutically effective amount of 12.5 mg to 50 mg (single dose, suitable for children and adults, total daily dose not exceeding 150 mg to 300 mg). In some embodiments, the oral antihistamine is cetirizine, with a therapeutically effective amount of 5 mg to 10 mg (daily dose). In some embodiments, the oral antihistamine is loratadine, with a therapeutically effective amount of 5 mg to 10 mg (daily dose). In some embodiments, the oral antihistamine is fexofenadine, with a therapeutically effective amount of 30 mg to 180 mg (daily dose).

In some embodiments, the medicine is used as a medicine for complementary therapy and can be used in combination with any other treatment for AD. In some embodiments, said any other treatment for AD is administration of a therapeutically effective amount of the topical steroid ointment. When administering the topical steroid ointment, the dosage is calculated in fingertip units (FTU). Adults are administered 0.5 FTU to 2.0 FTU per dose, which can cover a skin area from 50 to 200 square centimeters (cm²), and the frequency of application is 1 to 2 times per day; wherein the specific dosage and frequency of application are adjusted according to the severity of the patient's lesion area. 1 FTU is equal to a strip of ointment about 2.5 cm long squeezed from the tip of an adult's finger (about 0.5 g), enough to cover approximately 2 adult hand-sized skin areas (about 200 cm²). The usual dose for infants of 1 year old or younger is 1 FTU to 1.5 FTU per dose, which can be specifically adjusted based on the area and location of the lesion. The usual dose for children over 1 to 3 years old is 1.5 FTU to 3 FTU per dose, particularly adjusted based on the area and location of the lesion. The dose for children over 3 to 6 years old can be 1.5 FTU to 3.5 FTU per dose, particularly adjusted based on the area and location of the lesion. The dose for children over 6 to 10 years old can be 2 FTU to 5 FTU per dose, particularly adjusted based on the area and location of the lesion. The dose for children over 10 years old can be 2.8 FTU to 8 FTU per dose, particularly adjusted based on the area and location of the lesion. Adolescents over 12 years old may refer to adult dosage.

In some embodiments, said any other treatment for AD is administration of a therapeutically effective amount of oral antihistamines. The therapeutically effective amount of oral antihistamines is as described above.

In some embodiments, the food product further comprises food additives.

The composition comprising LMF provided by the present invention can significantly improve the SCORAD index of patients with AD, including reducing the lesion area and severity (objective indicators of the SCORAD index), and improving the itching sensation and insomnia symptoms caused by itching in patients with AD (subjective indicator of the SCORAD index). The present invention can also reduce the frequency of topical steroid ointment application and/or oral antihistamine application, even achieve the effect of not using any topical steroid ointments and/or oral antihistamines at all. At the same time, the composition of the present invention will not increase the biochemical indices of liver and kidney and simultaneously have effectiveness and safety. If the composition comprising LMF of the present invention can be administered in conjunction with the primary treatment for patients with AD, then it can effectively improve the symptoms and medication status of patients with AD, and it will not cause burden on the liver and kidney.

### BRIEF DESCRIPTION OF THE DRAWINGS

FIG. 1A is the change of lesion area of SCORAD index in the experimental group and the control group during a 16-week trial period;

FIG. 1B is the change of lesion severity of SCORAD index in the experimental group and the control group during a 16-week trial period;

FIG. 1C is the change of subjective indicator of SCORAD index in the experimental group and the control group during a 16-week trial period;

FIG. 1D is the change of total scores of SCORAD index in the experimental group and the control group during a 16-week trial period;

FIG. 2A is the change of frequency of topical steroid ointment application in the experimental group and the control group during a 16-week trial period;

FIG. 2B is the change of frequency of oral antihistamine application in the experimental group and the control group during a 16-week trial period;

FIG. 3A is the change of serum IgE concentration in the experimental group and the control group at Weeks 0, 12, and 16 of the trial;

FIG. 3B is the change of ESR in the experimental group and the control group at Weeks 0, 12, and 16 of the trial;

FIG. 3C is the change of serum CRP concentration in the experimental group and the control group at Weeks 0, 12, and 16 of the trial;

FIG. 3D is the change of eosinophil percentage in the experimental group and the control group at Weeks 0, 12, and 16 of the trial;

FIG. 4A is the change of CD4 T cell percentage in the experimental group and the control group at Weeks 0, 12, and 16 of the trial;

FIG. 4B is the change of CD8 T cell percentage in the experimental group and the control group at Weeks 0, 12, and 16 of the trial;

FIG. 4C is the change of serum IFN-γ concentration in the experimental group and the control group at Weeks 0, 12, and 16 of the trial;

FIG. 4D is the change of serum IL-4 concentration in the experimental group and the control group at Weeks 0, 12, and 16 of the trial;

FIG. 4E is the change of serum IL-10 concentration in the experimental group and the control group at Weeks 0, 12, and 16 of the trial;

FIG. 4F is the change of serum IL-12 concentration in the experimental group and the control group at Weeks 0, 12, and 16 of the trial;

FIG. 4G is the change of serum IL-13 concentration in the experimental group and the control group at Weeks 0, 12, and 16 of the trial;

FIG. 5A is the change of serum aspartate aminotransferase (AST) concentration in the experimental group and the control group at Weeks 0, 12, and 16 of the trial;

FIG. 5B is the change of serum alanine aminotransferase (ALT) concentration in the experimental group and the control group at Weeks 0, 12, and 16 of the trial; and

FIG. 5C is the change of serum creatinine (Cr) concentration in the experimental group and the control group at Weeks 0, 12, and 16 of the trial.

### DETAILED DESCRIPTION OF THE PREFERRED EMBODIMENTS

Through the following detailed description of the embodiments and the accompanying drawings, the purpose, advantages, and technical features of the present invention should be obvious.

### Clinical Trial of LMF as a Complementary Therapy in the Treatment of AD

Between March 2017 and July 2020, a randomized, double-blind, placebo-controlled clinical trial was conducted in Taoyuan Chang Gung Memorial Hospital, Taiwan, to study the clinical efficacy and safety of LMF as a complementary therapy for the treatment of AD. The study was approved by the Institutional Review Board (IRB) of Chang Gung Medical Foundation, with the IRB certification of 201601520A3C601, and conducted in accordance with the recommendations of the committee's clinical trial guidelines. Written consents were obtained from all participants.

The inclusion criteria for participants were as follows, wherein participants should meet the following conditions (A1) to (A4) at the same time:
(A1) Patients aged between 4 and 60 years, either male or female;
(A2) Patients having skin itching (or parental report of scratching or rubbing) accompanied by 3 or more of the following situations:
   a) A history of itchiness in skin creases (for example, front of the elbows, back of the knees, front of the ankles, and areas around the neck or eyes);
   b) A history of asthma and hay fever;
   c) A history of general dry skin in the past year;
   d) Childhood onset at the age of two years or below;
   e) Visible flexural dermatitis;
(A3) Patients with a SCORAD index ≥16; and
(A4) Patients who voluntarily agreed to participate and provided an informed consent.

The exclusion criteria for participants were as follows, wherein participants should be excluded when they met any of the conditions from (B1) to (B9):
(B1) Patients having other eczema diseases such as contact dermatitis or seborrheic dermatitis, as assessed by a dermatologist;
(B2) Patients having other skin diseases associated with itchy skin, as assessed by a dermatologist;
(B3) Patients who had been administered with any of the following within 1 month prior to registration: oral/intravenous steroids, leukotriene antagonists, immunosuppressants, systemic photochemotherapy, immunotherapy, allergen-specific immunotherapy, or Chinese herbal medicine;
(B4) Patients unable to use the test supplements as scheduled, or unable to cooperate filling the questionnaires and taking blood tests;
(B5) Patients having a history of allergy to fucoidan;
(B6) Patients having a history of hyperthyroidism;
(B7) Patients having a serious infection requiring hospitalization (for example, pneumonia, cellulitis, or sepsis), as assessed by a clinician;
(B8) Patients having severe organ dysfunction (for example, heart failure, liver failure, liver cirrhosis, or renal failure [eGFR <60 mL/min/1.73 m2]), as assessed by a clinician; and
(B9) Pregnant females, or females preparing for pregnancy, or females 4 in lactation.

A total of 98 participants were invited to participate in the above trials, wherein 7 participants did not meet the inclusion criteria and were excluded. Eligible participants (number (n)=91) were randomly assigned to either the experimental group (n=60) or the control group (n=31) at a 2:1 ratio.

Participants were randomly grouped, and participants, pharmacists, and researchers did not know what the group of participants were throughout the clinical trial.

In addition to receiving routine treatment of AD (including the administration of topical steroid ointments and oral antihistamines according to the clinician's instructions), participants were orally supplemented with LMF (experimental group) or placebo (control group) for 12 consecutive weeks, and observed for additional 4 weeks (a total of 16 weeks) after discontinuation of supplementation with LMF or placebo. During the trial, participants should record their daily medication, and they had been instructed to reduce the frequency of topical steroid ointment application and/or oral antihistamine application when the itching was tolerable and the lesion was stable.

The LMF used in the present invention was LMF powder (purchased from Hi-Q Marine Biotech International Ltd.), which was extracted and purified from Sargassum spp. and/or Laminaria japonica (also known as Saccharina japonica). In addition, the LMF used in the present invention was a product obtained by enzymatic hydrolysis of original fucoidan. The LMF of the present invention had a Mw of 3,000 Da or less (92.1% having a Mw of 3,000 Da or less), and it comprised fucose monomers modified by sulfate group, wherein the fucose content was 210.9±3.3 micromoles (μmol)/g, and the sulfate group content was 38.9±0.4% (weight in weight, w/w).

LMF and placebo were administered in a capsule form, wherein each capsule of experimental group had 500 mg of LMF, and each capsule of control group had 500 mg of microcrystalline cellulose as placebo. The capsules of experimental group and in control group had the same appearance. The participants in the experimental group took the capsules of experimental group orally twice a day before breakfast and before dinner for 12 weeks; the participants in the control group took the placebo capsules at the same frequency. Participants' supplementary doses were determined based on their body weights. For those weighing more than 40 kg, the supplementary dose was 4.0 g/day (four capsules each time, twice per day); for those weighing 20 kg to 40 kg, the supplementary dose was 3.0 g/day (three capsules each time, twice per day); for those weighing 10 kg to less than 20 kg, the supplementary dose was 2.0 g/day (two capsules each time, twice per day); for those weighing less than 10 kg, the supplementary dose was 1.0 g/day (one capsule each time, twice per day).

Each participant was scheduled for four outpatient visits to assess the severity of symptoms: Week 0 (before LMF/placebo supplementation), Week 6 (6 weeks after LMF/placebo supplementation), Week 12 (12 weeks after LMF/placebo supplementation, end of supplementation), and Week 16 (end of trial). During the outpatient visits at Weeks 0, 6, 12, and 16, physicians used the SCORAD index to evaluate the severity of AD symptoms in each participant. The SCORAD index scoring items include the area and severity of AD lesions (the above two used as objective indicators), and itching and insomnia (as one subjective indicator, including insomnia symptoms caused by itching). In addition, these three indicators are added as a total score. Participants should report any symptoms or discomfort to the researchers, if any.

In addition, the blood samples of participants were collected at three time points of Weeks 0, 12 and 16 respectively and sent to Department of Laboratory Medicine in Taoyuan Chang Gung Memorial Hospital, Taiwan. The complete blood count and biochemical indices were tested, and the values of the following markers were specifically recorded:
Immunoglobulin E (IgE) concentration: the unit is kilounits per liter (KU/L);
Erythrocyte sedimentation rate (ESR): the unit is millimeters per hour (mm/h);
C-reactive protein (CRP) concentration: the unit is milligrams per liter (mg/L);
Eosinophil percentage: the unit is %;
White blood cell (WBC) count: the unit is 10³ cells per microliter (10³/μL);
Creatinine (Cr) concentration: the unit is milligrams per deciliter (mg/dL);
Aspartate aminotransferase (AST) concentration: the unit is unit per liter (U/L);
Alanine aminotransferase (ALT) concentration: the unit is U/L.

Antibodies against CD4 and CD8 (purchased from BD Biosciences, San Jose, CA, USA) were used to label blood cells, and the percentages of CD4⁺ T cells and CD8⁺ T cells (both units are %) were calculated using a BD FACSCanto™ II clinical flow cytometer (BD Biosciences) equipped with FASDiva software.

Quantikine kit (purchased from R&D Systems, Minneapolis, MN, USA) was also used to measure the following AD-related cytokines with enzyme-linked immunosorbent assay (ELISA) according to the manufacturer's instructions:
Interferon gamma (IFN-γ): the unit is KU/L;
Interleukin-4 (IL-4): the unit is picogram per milliliter (pg/mL);
Interleukin-10 (IL-10): the unit is pg/mL;
Interleukin-12 (IL-12): the unit is pg/mL;
Interleukin-13 (IL-13): the unit is pg/mL.

Data are denoted by mean±standard deviation (SD). Relevant statistics were performed using SPSS version 29.0.0.0 (SPSS Inc., Chicago, IL, USA) software, using the paired sample t-test to analyze the changes in SCORAD index, topical steroid ointment application, and oral antihistamine application at Weeks 6, 12, and 16 relatively to the baseline (Week 0). Changes of markers relatively to the baseline (Week 0) in other blood samples were also analyzed using the Wilcoxon signed-rank test. Two-tailed p<0.05 was considered statistically significant.

In all the participants, 9 participants were unable to complete the 16-week trial (including 6 in the experimental group and 3 in the control group), including: 2 participants lost contact during the follow-up period (1 in the experimental group and 1 in the control group), 5 participants had poor compliance with supplementary medications (4 in the experimental group and 1 in the control group), 1 participant self-injected steroids at another hospital (experimental group), and 1 participant had difficulty swallowing capsules due to facial nerve problem (control group). Finally, the results of 82 participants completing the 16-week trial were analyzed, including 54 participants in the experimental group and 28 participants in the control group.

Before the start of the trial, the basic information and the results of the aforementioned 82 participants are as shown in Table 1 below.

TABLE 1

| | Experimental | Control group | |
|---|---|---|---|
| | group (n = 54) | (n = 28) | |
| Age | Patient number | Patient number | p value |
| 4 to 10 years old | 14 | 8 | 0.946 |
| 11 to 20 years old | 13 | 9 | |
| 21 to 30 years old | 12 | 4 | |
| 31 to 40 years old | 8 | 4 | |
| 41 to 50 years old | 5 | 2 | |
| 51 to 60 years old | 2 | 1 | |

| | Patient number | Patient number | |
|---|---|---|---|
| Gender | (male/female) | (male/female) | p value |
| | 27/27 | 11/17 | 0.356 |

| SCORAD index | Mean ± SD | Mean ± SD | p value |
|---|---|---|---|
| Lesion area | 22.52 ± 21.52 | 17.61 ± 13.99 | 0.278 |
| Lesion severity | 7.03 ± 2.49 | 6.70 ± 2.40 | 0.565 |
| Subjective indicator | 10.66 ± 4.90 | 10.63 ± 4.65 | 0.979 |
| Total score | 39.78 ± 14.28 | 37.61 ± 13.20 | 0.507 |

| Application Frequency | | | |
|---|---|---|---|
| of commonly known | | | |
| medicine | Mean ± SD | Mean ± SD | p value |
| Topical steroid ointment | 3.75 ± 3.49 | 2.93 ± 3.46 | 0.317 |
| (days/week) | | | |
| Oral antihistamine | 1.91 ± 3.11 | 1.04 ± 2.53 | 0.185 |
| (days/week) | | | |

| Other markers | Mean ± SD | Mean ± SD | p value |
|---|---|---|---|
| IgE (KU/L) | 2216.6 ± 401.9 | 2274.2 ± 528.9 | 0.715 |
| ESR (mm/h) | 7.66 ± 0.71 | 8.18 ± 0.92 | 0.464 |
| CRP (mg/L) | 1.24 ± 0.35 | 1.08 ± 0.30 | 0.616 |
| Eosinophils (%) | 6.04 ± 0.76 | 6.88 ± 0.94 | 0.083 |
| WBC count (10³/μL) | 7.64 ± 0.29 | 8.26 ± 0.27 | 0.275 |
| AST (U/L) | 27.74 ± 1.81 | 24.96 ± 1.33 | 0.220 |
| ALT (U/L) | 21.15 ± 2.08 | 16.79 ± 1.75 | 0.288 |
| Cr (mg/dL) | 0.62 ± 0.03 | 0.59 ± 0.03 | 0.966 |

From Table 1, it was found that before the start of the trial, the participants in the experimental group and the control group had no significant differences in age, gender, SCORAD index at the beginning of the trial, the application frequency of commonly known medicine (including topical steroid ointments and oral antihistamines), and the values of several serological markers.

### The Impact of LMF on the SCORAD Index

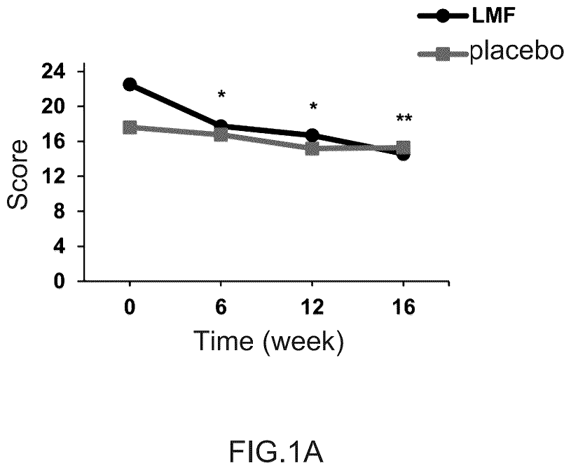

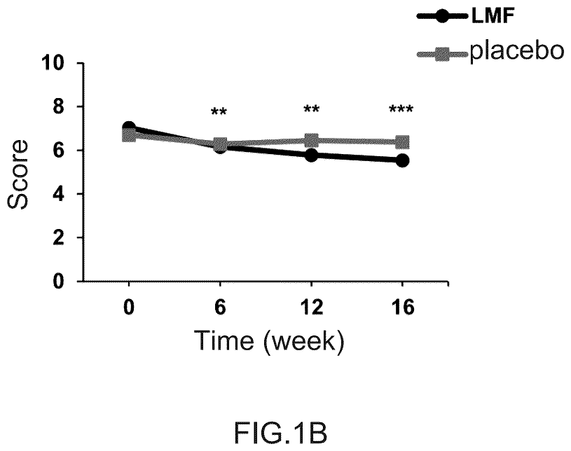

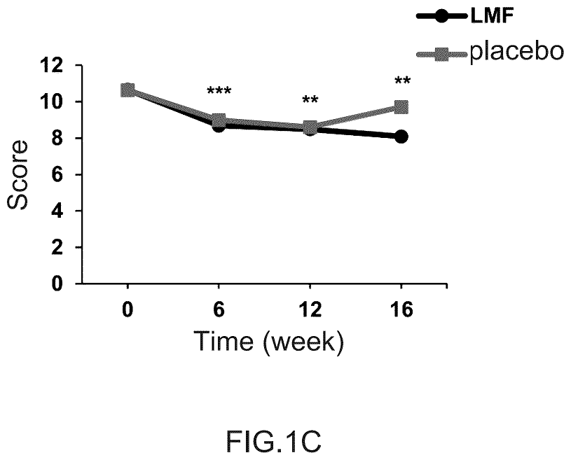

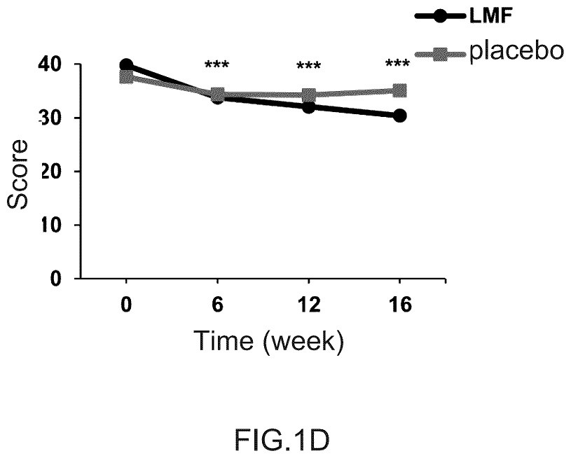

The SCORAD index was used to measure the impact of LMF supplementation on the severity of AD symptoms after 3 months (Week 12), including the changes of the objective indicators (area and severity of AD lesions) and the subjective indicator (itching and insomnia), as shown in Table 2 below and FIGS. 1A to 1D. In Table 2, * represents p<0.05, ** represents p <0.01, and *** represents p<0.001, indicating the results compared with the baseline (Week 0).

TABLE 2

| | | Week 0 | Week 6 | Week 12 | Week 16 |
|---|---|---|---|---|---|
| Experimental | Lesion area | 22.52 ± 21.52 | 17.74 ± 15.10* | 16.69 ± 15.33* | 14.57 ± 11.00** |
| group | Lesion | 7.03 ± 2.49 | 6.15 ± 2.49** | 5.78 ± 2.66** | 5.54 ± 2.41*** |
| (n = 54) | severity | | | | |
| | Subjective | 10.66 ± 4.90 | 8.69 ± 4.77*** | 8.48 ± 5.33** | 8.10 ± 5.05** |
| | indicator | | | | |
| | Total score | 39.78 ± 14.28 | 33.77 ± 13.48*** | 32.06 ± 14.58*** | 30.41 ± 12.32*** |
| Control | Lesion area | 17.61 ± 13.99 | 16.75 ± 16.23 | 15.18 ± 14.53 | 15.29 ± 15.21 |
| group | Lesion | 6.70 ± 2.40 | 6.29 ± 2.74 | 6.45 ± 2.51 | 6.38 ± 2.56 |
| (n = 28) | severity | | | | |
| | Subjective | 10.63 ± 4.65 | 8.99 ± 5.81 | 8.60 ± 5.34 | 9.71 ± 5.42 |
| | indicator | | | | |
| | Total score | 37.61 ± 13.20 | 34.35 ± 16.34 | 34.22 ± 14.95 | 34.22 ± 14.95 |

From Table 2 and FIGS. 1A and 1B, it was found that in the experimental group, the objective indicators of both lesion area and lesion severity significantly decreased: the lesion area score of the SCORAD index could be reduced by 25.9% after 12 weeks of administration, and continued to decrease after discontinuation of medication, reaching a reduction effect of 35.3%; the lesion severity score of the SCORAD index could be reduced by 18.5% after 12 weeks of administration, and continued to decrease after discontinuation of medication, reaching a reduction effect of 21.2% (all having statistically significant differences). In addition, it can be seen from Table 2 and FIGS. 1C and 1D that the subjective indicator and total score of the SCORAD index decreased significantly at Week 6 and Week 12: the subjective indicator score of the SCORAD index could be reduced by 20.5% after 12 weeks of administration, and continued to decrease after discontinuation of medication, reaching a reduction effect of 24.0%; while the total score of the SCORAD index could be reduced by 19.4% after 12 weeks of administration, and continued to decrease after discontinuation of medication, achieving a reduction effect of 23.6% (all having statistically significant differences). Even one month after discontinuation of the administration of LMF (Week 16), the SCORAD index of the above-mentioned experimental group continued to decrease. However, in the control group, there were no significant changes in each SCORAD index before and after using placebo. Although the total score of SCORAD index decreased, there is no statistically significant difference. This shows that LMF can indeed significantly improve the SCORAD index (including subjective and objective indicators) in patients with AD.

### Impact of LMF on the Administration of Topical Steroid Ointments and Oral Antihistamines

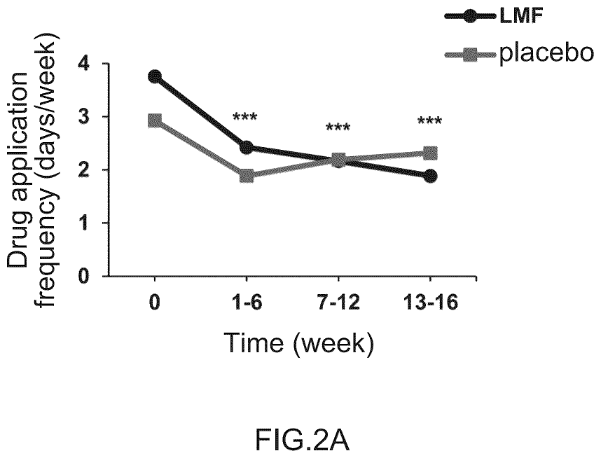

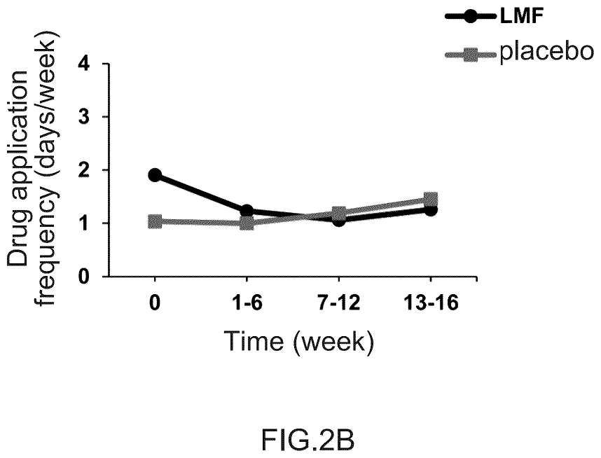

In addition to changes in the SCORAD index, the impact of LMF on the administration of topical steroid ointments and oral antihistamines was further observed. The participant's frequency of topical steroid ointment application and oral antihistamine application was recorded in terms of days of use per week (days/week), as shown in Table 3 below and FIGS. 2A to 2B. In Table 3, *** represents p<0.001, indicating the results compared with the baseline (Week 0).

TABLE 3

| | | | Weeks | Weeks | Weeks |
|---|---|---|---|---|---|
| | | Week 0 | 0 to 6 | 7 to 12 | 13 to 16 |
| Experimental | Topical steroid | 3.75 ± 3.49 | 2.42 ± 2.85*** | 2.16 ± 2.83*** | 1.89 ± 2.86*** |
| group | ointment | | | | |
| (n = 54) | (days/week) | | | | |
| | Oral | 1.91 ± 3.11 | 1.23 ± 2.21 | 1.06 ± 2.27 | 1.26 ± 2.46 |
| | antihistamine | | | | |
| | (days/week) | | | | |
| Control | Topical steroid | 2.93 ± 3.46 | 1.89 ± 2.63 | 2.19 ± 2.84 | 2.31 ± 3.09 |
| group | ointment | | | | |
| (n = 28) | (days/week) | | | | |
| | Oral | 1.04 ± 2.53 | 1.00 ± 2.00 | 1.19 ± 2.48 | 1.45 ± 2.58 |
| | antihistamine | | | | |
| | (days/week) | | | | |

From Table 3 and FIGS. 2A and 2B, it was found that at Week 0, there was no statistically significant difference in the frequency of commonly known medicines (including topical steroid ointments and oral antihistamines) in the experimental group and the control group. However, in the experimental group, the frequency of topical steroid ointment application gradually decreased, and this effect was retained even after discontinuation of LMF (Weeks 13 to 16): the frequency of topical steroid ointment application could be reduced by 42.4% after 12 weeks of administration, and continued to decrease after discontinuation of medication, reaching a reduction effect of 49.6% (with a statistically significant difference); the frequency of oral antihistamine application also decreased: the frequency of oral antihistamine application could be reduced by 44.5% after 12 weeks of administration, and the effect was still reduced by 34.0% after 4 weeks of discontinuation (without statistically significant difference, wherein the p values of Weeks 1-6 and Weeks 7-12 compared to the baseline (Week 0) are 0.074 and 0.057 respectively). In contrast, in the control group, there was no statistically significant difference in the frequency of topical steroid ointment application and/or oral antihistamine application before and after placebo supplementation, and the frequency of application of oral antihistamines increased by 39.4%; therefore, LMF still had the effect to a certain extent in reducing the frequency of oral antihistamines administration.

Also, since patients may administer topical steroid ointment and oral antihistamine at the same time, they may also administer different medicines at different stages. Therefore, topical steroid ointment application alone (T only), oral antihistamine application alone (A only), both (simultaneous administration of topical steroid ointment and oral antihistamine), and neither (none of topical steroid ointment and oral antihistamine) in the experimental group and the control group were further observed, and the results are shown in Table 4. To facilitate comparison, the numbers of people are also presented as percentages, with the denominator of the experimental group being 54 (persons) and the denominator of the control group being 28 (persons).

TABLE 4

| | | | | Weeks | | Weeks | | Weeks | |
|---|---|---|---|---|---|---|---|---|---|
| | | Week 0 | | 0 to 6 | | 7 to 12 | | 13 to 16 | |
| | | n | % | n | % | n | % | n | % |
| Experimental | T only | 12 | 22.2% | 15 | 27.8% | 11 | 20.4% | 7 | 13.0% |
| group | A only | 5 | 9.3% | 8 | 14.8% | 6 | 11.1% | 5 | 9.3% |
| (n = 54) | Both | 18 | 33.3% | 17 | 31.5% | 16 | 30.0% | 14 | 25.9% |
| | Neither | 19 | 35.2% | 14 | 25.9% | 21 | 38.9% | 28 | 51.9% |
| Control | T only | 8 | 28.6% | 12 | 42.9% | 11 | 39.3% | 7 | 25% |
| group | A only | 1 | 3.6% | 1 | 3.6% | 3 | 10.7% | 3 | 10.7% |
| (n = 28) | Both | 6 | 21.4% | 7 | 25% | 6 | 21.4% | 7 | 25% |
| | Neither | 13 | 46.4% | 8 | 28.6% | 8 | 28.6% | 11 | 39.3% |

The overall medication trend of the participants can be observed from the data in Table 3 and Table 4. As seen from Table 4, compared with the control group, LMF could reduce the proportion of people administering topical steroid ointment alone (from 22.2% to 13.0%, a decrease of 9.2%), and the proportion of people administering both (topical steroid ointments and oral steroid ointments) (from 33.3% to 25.9%, a decrease of 7.4%), while the group administering oral antihistamines alone remained at the same level (both 9.3%, but the control group of oral antihistamines alone increased from 3.6% to 10.7%, an increase of 7.1%). Overall, LMF could significantly increase the proportion of people no longer administering steroids or antihistamines (from 35.2% to 51.9%, an increase of 16.7%), so it is indeed an effective complementary therapy. It should also be noted that the data from Weeks 13 to 16 of the experimental group shows that even if the participants in the experimental group stopped taking LMF, the proportion of medicine administration (topical steroid ointments and/or oral antihistamines) did not rebound immediately. On the contrary, they continued to decrease, indicating LMF could still achieve effect to a certain extent 1 month after administration, and the effect of medicine could be maintained for a period of time.

The impact of LMF on other AD-related indicators

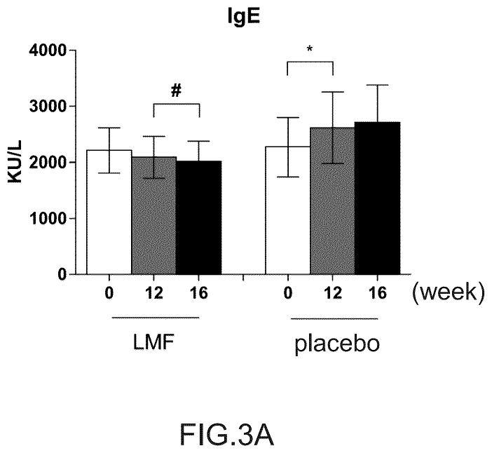

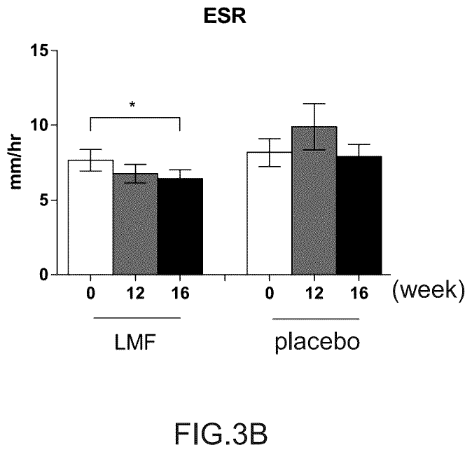

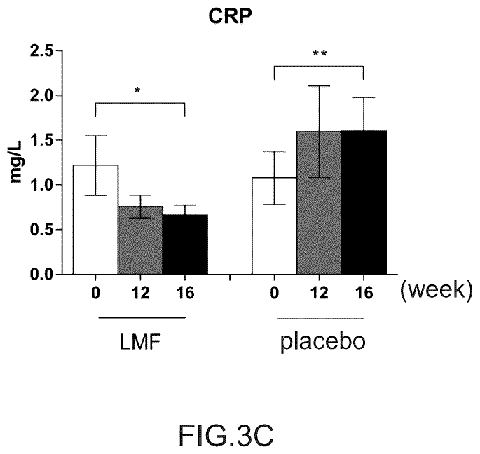

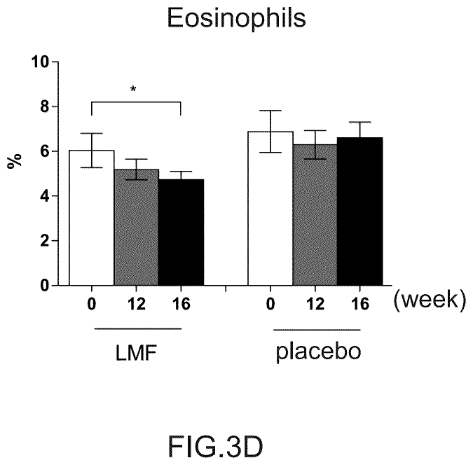

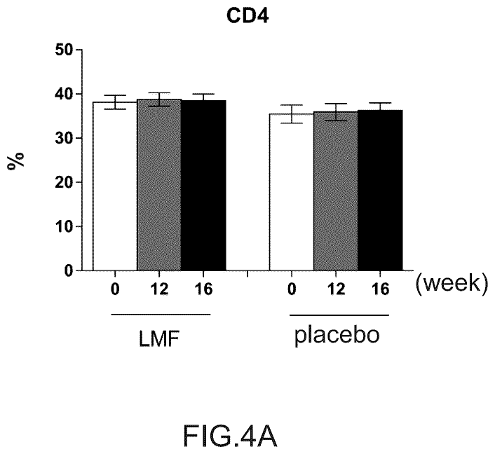

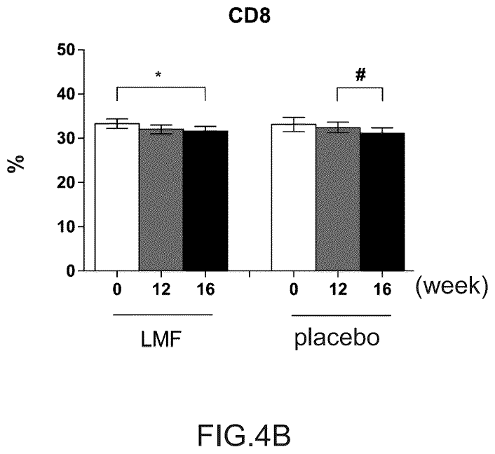

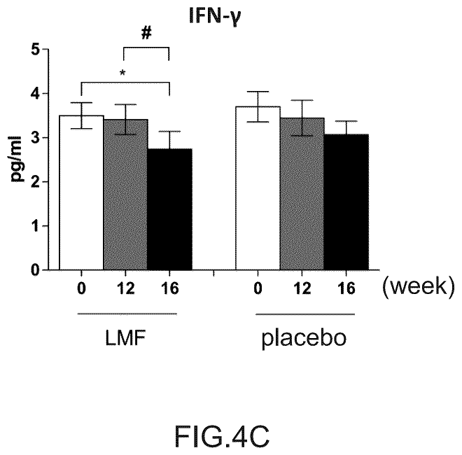

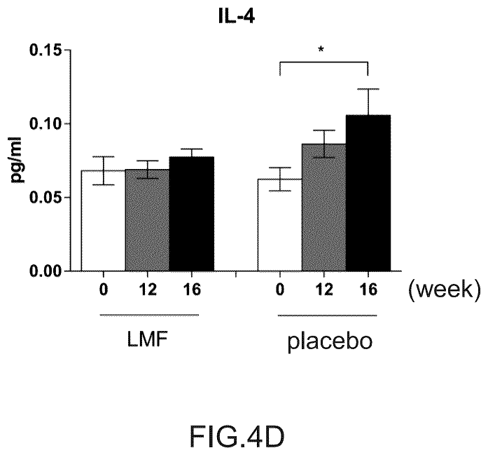

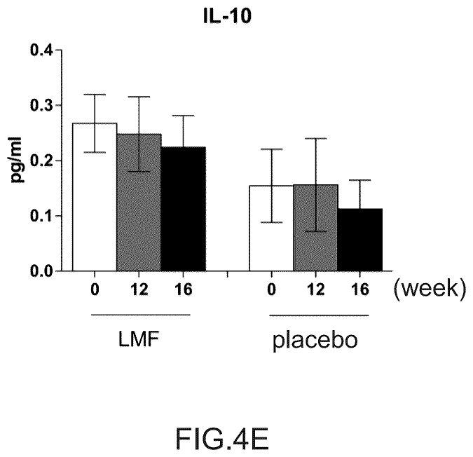

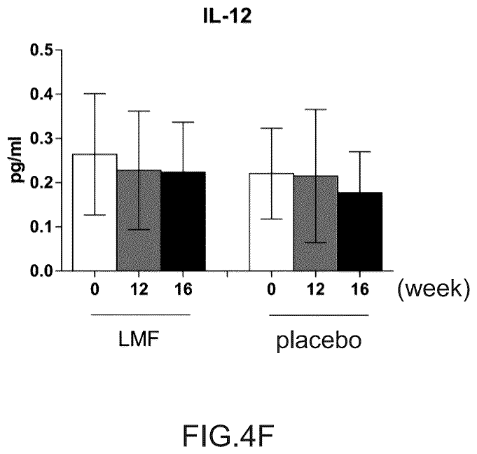

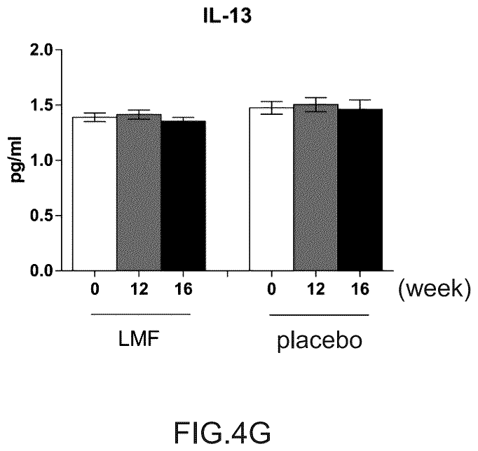

In addition, the changes of several AD-related immune parameters were recorded before and after the trial (Week 0 and Week 12) and four weeks after discontinuation of the administration of LMF (Week 16), as shown in Table 5 below. The changes in four serological markers are shown in FIGS. 3A to 3D, and the changes in other cytokine indicators are shown in FIGS. 4A to 4G. In Table 5, * represents p<0.05, indicating the results compared with the baseline (Week 0); # represents p<0.05, indicating the results compared with the data of Week 12.

TABLE 5

| | | Week 0 | Week 12 | Week 16 |
|---|---|---|---|---|
| Experimental | IgE (KU/L) | 2216.6 ± 401.9 | 2090.2 ± 370.8 | 2019.6 ± 359.7# |
| group | ESR (mm/h) | 7.66 ± 0.71 | 6.78 ± 0.63 | 6.44 ± 0.61* |
| (n = 54) | CRP (mg/L) | 1.24 ± 0.35 | 0.77 ± 0.13 | 0.67 ± 0.12* |
| | Eosinophils (%) | 6.04 ± 0.76 | 5.18 ± 0.46 | 4.7 ± 0.37* |
| | WBC count (10³/μL) | 7.64 ± 0.29 | 7.62 ± 0.29 | 7.48 ± 0.31 |
| | IFN-γ (pg/mL) | 3.499 ± 0.293 | 3.411 ± 0.343 | 2.741 ± 0.402*# |
| | IL-4 (pg/mL) | 0.068 ± 0.010 | 0.069 ± 0.006 | 0.077 ± 0.006 |
| | IL-10 (pg/mL) | 0.267 ± 0.052 | 0.248 ± 0.068 | 0.224 ± 0.057 |
| | IL-12 (pg/mL) | 0.252 ± 0.129 | 0.217 ± 0.126 | 0.214 ± 0.106 |
| | IL-13 (pg/mL) | 1.335 ± 0.066 | 1.356 ± 0.070 | 1.298 ± 0.064 |
| | CD4⁺ T cells (%) | 38.16 ± 1.55 | 38.75 ± 1.53 | 38.51 ± 1.46 |
| | CD8⁺ T cells (%) | 33.33 ± 1.05 | 32.0 ± 01.02 | 31.67 ± 1.01* |
| Control | IgE (KU/L) | 2274.2 ± 528.9 | 2619.7 ± 633.7* | 2716.6 ± 663.6 |
| group | ESR (mm/h) | 8.18 ± 0.92 | 9.89 ± 1.55 | 7.89 ± 0.82 |
| (n = 28) | CRP (mg/L) | 1.08 ± 0.30 | 1.60 ± 0.51 | 1.60 ± 0.38** |
| | Eosinophils (%) | 6.88 ± 0.94 | 6.29 ± 0.64 | 6.60 ± 0.71 |
| | WBC count (10³/μL) | 8.26 ± 0.27 | 8.25 ± 0.39 | 8.44 ± 0.44 |
| | IFN-γ (pg/mL) | 3.700 ± 0.345 | 3.444 ± 0.404 | 3.068 ± 0.307 |
| | IL-4 (pg/mL) | 0.063 ± 0.008 | 0.086 ± 0.009 | 0.106 ± 0.018* |
| | IL-10 (pg/mL) | 0.154 ± 0.066 | 0.156 ± 0.084 | 0.113 ± 0.052 |
| | IL-12 (pg/mL) | 0.220 ± 0.103 | 0.215 ± 0.151 | 0.178 ± 0.092 |
| | IL-13 (pg/mL) | 1.475 ± 0.058 | 1.503 ± 0.064 | 1.461 ± 0.085 |
| | CD4⁺ T cells (%) | 35.44 ± 2.04 | 35.92 ± 1.93 | 36.25 ± 1.78 |
| | CD8⁺ T cells (%) | 33.16 ± 1.63 | 32.46 ± 1.22 | 31.09 ± 1.31# |

From Table 5 and FIGS. 3A to 3D, in the experimental group, the values of AD-related immune parameters (IgE concentration, ESR, CRP, and eosinophil percentage) were reduced after 12 weeks of administration, and continued to decrease after 4 weeks (Week 16) of medication discontinuation, indicating LMF could indeed change the above immune parameters and improve the condition of AD. The IgE concentration in serum could be reduced by 5.7% after 12 weeks of administration, and continued to decrease after discontinuation of medication, reaching a reduction effect of 8.9%; the ESR could be reduced by 11.5% after 12 weeks of administration, and continued to decrease after discontinuation of medication, reaching a reduction effect of 15.9%; the CRP concentration in serum could be reduced by 37.9% after 12 weeks of administration, and continued to decrease after discontinuation of medication, reaching a reduction effect of 46.0%; the eosinophil percentage could be reduced by 14.2% after 12 weeks of administration, and continued to decrease after discontinuation of medication, reaching a reduction effect of 22.2% (the groups above all having statistically significant differences). However, in the control group, the IgE concentration and CRP increased, and the values of ESR and eosinophil percentage remained, without statistically significant differences.

In Table 5 and FIGS. 4A and 4B, the CD4⁺ T cell percentage in the experimental group and the control group remained at Weeks 0, 12, and 16 (with no statistically significant difference); the CD8⁺ T cell percentage in the experimental group decreased, and the value had a statistically significant difference.

Although CD8⁺ T cells are responsible for virus clearance and immune regulation in the immune system, the overactivation of these cells in patients with AD may promote inflammatory responses. The treatment method of the present invention can reduce the proportion of CD8⁺ T cells and thus regulate the patient's immune response, reducing the local inflammatory burden on the skin, thereby helping to relieve symptoms of AD and improve skin barrier function.

In Table 5 and FIG. 4C, the IFN-γ concentration in serum continued to decrease significantly in the experimental group: the IFN-γ concentration in serum could be reduced by 2.5% after 12 weeks of administration, and continued to decrease after discontinuation of medication, reaching a reduction effect of 21.7% (with statistically significant differences). In addition, in Table 5 and FIG. 4C, the IL-4 concentration in serum increased significantly in the control group: the IL-4 concentration in serum could be elevated by 36.5% after 12 weeks of administration, and continued to increase after discontinuation of medication, reaching an elevating effect of 68.3%; in contrast, the IL-4 concentration in serum in the experimental group remained: the IL-4 concentration in serum remained after 12 weeks of administration, and only increased by 13.2% after discontinuation of medication; therefore, it was found that the serum IL-4 in patients with AD would increase, and LMF could reduce the serum IL-4 content (all having statistically significant differences in the experimental group and the control group). However, in Table 5 and FIGS. 4E to 4G, in the experimental group and the control group, the concentrations of other cytokines (including IL-10, IL-12, and IL-13) were roughly the same at Weeks 0, 12, and 16, and there was no statistically significant difference.

It can be found from the above that LMF can reduce the AD-related immune parameters including IgE, ESR, CRP, eosinophil percentage, IFN-γ in the serum of patients with AD, and reduce the increase of IL-4, and has a positive effect on controlling the condition of patients.

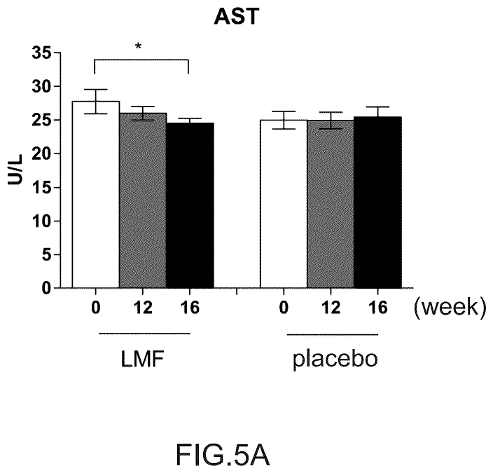

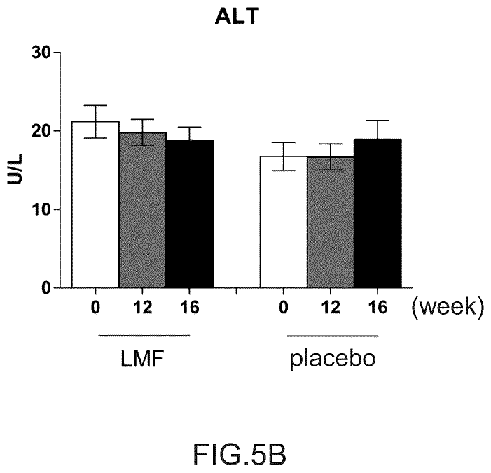

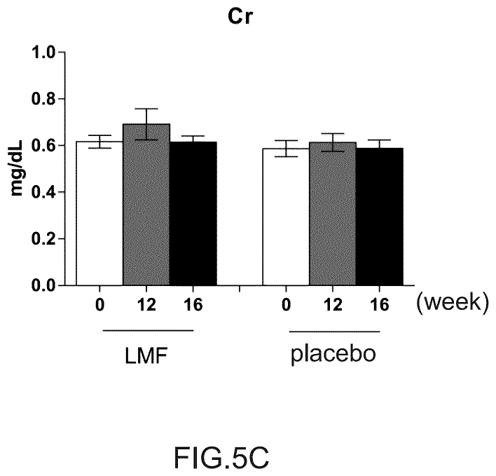

The changes in biochemical indices of liver and kidney functions were also recorded before and after the trial (Weeks 0 and 12) and 4 months after discontinuation of the administration of LMF (Week 16), as shown in FIGS. 5A to 5C. In FIG. 5A, * represents p<0.05, indicating the results compared with the baseline (Week 0). FIGS. 5A to 5C show that liver and kidney functions before and after supplementation with LMF or placebo in the experimental group and the control group were all in normal ranges, so LMF will not have negative impact on liver and kidney function of participants and have safety.

In summary, the composition comprising LMF provided in the present invention can significantly improve the subjective and objective indicators of the SCORAD index in patients with AD, reduce the frequency of topical steroid ointment application and/or oral antihistamine application, significantly increase the number of people without the need of topical steroid ointment application and/or oral antihistamine application, and reduce AD-related immune parameters, and it indeed has good effect. In addition, when used in treatments for AD, the composition of the present invention will not increase the biochemical indices of liver and kidney and have safety.

What is claimed is:

1. A method for improving atopic dermatitis, comprising administering to a subject in need thereof a composition comprising low-molecular-weight fucoidan, wherein the weight-average molecular weight of the low-molecular-weight fucoidan is 3,000 Da or less.

2. The method as claimed in claim 1, wherein an effective amount of the low-molecular-weight fucoidan is: 4.0 g/day for the subject weighing more than 40 kg; 3.0 g/day for the subject weighing 20 kg to 40 kg; 2.0 g/day for the subject weighing 10 kg to less than 20 kg; and 1.0 g/day for the subject weighing less than 10 kg.

3. The method as claimed in claim 1, wherein the composition reduces the total score of SCORAD index by 15% or more.

4. The method as claimed in claim 1, wherein the administering step is administering a medicine or food product comprising the composition comprising the low-molecular-weight fucoidan and at least one pharmaceutically acceptable excipient.

5. The method as claimed in claim 1, wherein the medicine is in an oral form.

6. The method as claimed in claim 5, wherein the composition further comprises a therapeutically effective amount of oral antihistamines.

7. The method as claimed in claim 1, wherein the medicine is used as a medicine for complementary therapy and is useable in combination with any other treatment for atopic dermatitis.

8. The method as claimed in claim 7, wherein said any other treatment for atopic dermatitis is administration of a therapeutically effective amount of topical steroid ointments.

9. The method as claimed in claim 7, wherein said any other treatment for atopic dermatitis is administration of a therapeutically effective amount of oral antihistamines.

10. The method as claimed in claim 1, wherein the food product further comprises food additives.

11. The method as claimed in claim 4, wherein the food product further comprises food additives.
